(19) United States
(12) Patent Application Publication
(10) Pub. No.: US 2024/0074484 A1
(43) Pub. Date: Mar. 7, 2024

(54) ARTICLE

(71) Applicant: RAI Strategic Holdings Inc., Winston-Salem, NC (US)

(72) Inventors: Michael Foster Davis, Winston-Salem, NC (US); John Will Caraway, Winston-Salem, NC (US)

(21) Appl. No.: 17/754,672

(22) PCT Filed: Oct. 8, 2020
(86) PCT No.: PCT/IB2020/059480

§ 371 Date: Apr. 8, 2022

Related U.S. Application Data
(60) Provisional application No. 62/912,974, filed on Oct. 9, 2019.

(51) Int. Cl. A24B 15/167; A24B 15/30; A24F 40/10; A24F 40/42; A24F 40/44

(52) U.S. Cl. CPC A24B 15/167; A24B 15/302; A24F 40/10; A24F 40/42; A24F 40/44

(57) ABSTRACT

An article for use with an aerosol provision system, the article comprising a store comprising an aerosolizable material and a wick formed from a sintered material, wherein the aerosolizable material comprises at least one cannabinoid and at least one carrier constituent.

### PRIORITY CLAIM

The present application is a National Phase entry of PCT Application No. PCT/IB2020/059480, filed Oct. 8, 2020, which claims benefit of U.S. Provisional Application No. 62/912,974, filed Oct. 9, 2019, each of which is hereby fully incorporated herein by reference.

### TECHNICAL FIELD

The present disclosure relates to an article comprising an aerosolizable material, as well as containers and systems comprising and using said article.

### BACKGROUND

Aerosol delivery systems which generate an aerosol for inhalation by a user are known in the art. Such systems typically comprise an aerosol generator which is capable of converting an aerosolizable material into an aerosol. In some instances, the aerosol generated is a condensation aerosol whereby an aerosolizable material is heated to form a vapor which is then allowed to condense into an aerosol. In other instances, the aerosol generated is an aerosol which results from the atomization of the aerosolizable material. Such atomization may be brought about mechanically, e.g. by subjecting the aerosolizable material to vibrations so as to form small particles of material that are entrained in airflow. Alternatively, such atomization may be brought about electrostatically, or in other ways, such as by using pressure etc.

Depending on the constituents of the aerosolizable material that are to be provided to a user, it may be preferable to formulate the aerosolizable material in a certain way. For example, it may be preferable to formulate the aerosolizable material so as to produce an aerosol with a particular profile. It may also be preferable to formulate the aerosolizable material so as to ensure the aerosolizable material meets certain standards of quality, consistency and the like.

It would thus be desirable to provide an aerosolizable material that is formulated so as to be acceptable to a user.

### SUMMARY

In one aspect there is provided an article for use with an aerosol provision system, the article comprising a store comprising an aerosolizable material and a wick formed from a sintered material, wherein the aerosolizable material comprises at least one cannabinoid and at least one carrier constituent.

For example, the article comprises a store for receiving the aerosolizable material, an aerosol generating component, an aerosol generating area, and/or a mouthpiece. Typically, the store for receiving an aerosolizable material will allow for the article to be refilled with aerosolizable material as the aerosolizable material is depleted during use. The aerosol generating component is generally a heater. An airflow pathway typically extends through the article (optionally via an aerosol delivery device) to an outlet. The pathway is oriented such that generated aerosol is entrained in the airflow such that it can be delivered to the outlet for inhalation by a user.

The article can be used with an aerosol delivery device so as to form an aerosol delivery system. The aerosol delivery device typically comprises a power source and a controller. In some instances, the aerosol delivery device will contain the aerosol generating component. During operation of the aerosol delivery device, the controller will determine that a user has initiated a request for the generation of an aerosol. This could be done via a button on the device which sends a signal to the controller that the aerosol generator should be powered. Alternatively, a sensor located in or proximal to the airflow pathway could detect airflow through the airflow pathway and convey this detection to the controller. A sensor may also be present in addition to the presence of a button, as the sensor may be used to determine certain usage characteristics, such as airflow, timing of aerosol generation etc.

Aerosolizable material is transferred from the store for receiving an aerosolizable material to the aerosol generating component via a wick. In particular, the wick is formed a sintered material.

The sintered material may comprise sintered ceramic, sintered metal fibers/powders, or a combination of the two. Further, the sintered wick may have deposited thereon/embedded therein an electrically resistive heater. Such an electrically resistive heater would typically be formed from a metal.

Alternatively, the sintered material may have such electrical properties such that when a current is passed there through, it is heated. Thus, the aerosol generating component and the wick may be considered to be integrated. In some embodiments, the aerosol generating component and the wick are formed from the same material and form a single component.

Cannabinoids are typically not soluble in water. They therefore require the use of other solvents so as to allow them to be prepared in liquid form. This can generally lead to liquids of higher viscosity. Due to the relatively higher viscosity of such liquids, an aerosolizable material which is formed from said liquid and which is to be aerosolized in an aerosol provision system may suffer from problems during transfer of the aerosolizable material from the store where it is held to where it will be aerosolized. In particular, when the flow rate of the aerosolizable material is low (perhaps due to its viscosity, it may be that there is insufficient flow to the heater. This can mean that there is insufficient aerosolizable material present in the proximity of the heater to receive energy from the heater. This “excess” energy results in the wick becoming hotter than it would otherwise be had there been a sufficient flow of aerosolizable material. A sintered wick may be ameliorate some of these problems. Sintered materials are generally more heat stable than other wicking components, such as cotton. Thus, the use of a sintered wick with an aerosolizable material comprising at least one cannabinoid can be advantageous.

Thus, in a further aspect there is provided an aerosol provision system comprising an aerosol provision device and an article as defined herein.

These aspects and other aspects will be apparent from the following detailed description. In this regard, particular sections of the description are not to be read in isolation from other sections.

### BRIEF DESCRIPTION OF THE DRAWINGS

Various embodiments will now be described in detail by way of example only with reference to the accompanying drawings in which:

FIG. 1— Provides a schematic overview of an article, aerosol delivery device and system as described herein.

### DETAILED DESCRIPTION OF THE DRAWINGS

In one aspect there is provided an article for use with an aerosol provision system, the article comprising a store comprising an aerosolizable material and a wick formed from a sintered material, wherein the aerosolizable material comprises at least one cannabinoid and at least one carrier constituent.

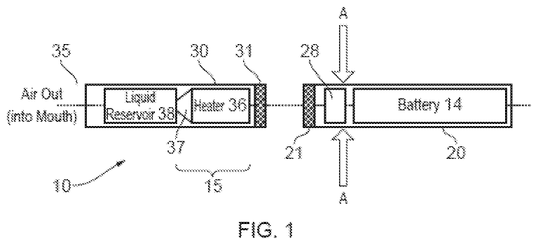

FIG. 1 is a highly schematic diagram (not to scale) of an example aerosol provision system, such as an e-cigarette 10, to which embodiments are applicable. The e-cigarette has a generally cylindrical shape, extending along a longitudinal axis indicated by a dashed line (although aspects of the invention are applicable to e-cigarettes configured in other shapes and arrangements), and comprises two main components, namely an aerosol provision device 20 and an article 30.

The article 30 includes a store for aerosolizable material (source liquid) 38 containing an aerosolizable material (source liquid) from which an aerosol is to be generated. The article 30 further comprises an aerosol generating component (heating element or heater) 36 for heating aerosolizable material to generate the aerosol. A wicking element or component or wick 37 is provided to deliver aerosolizable material from the store 38 to the heating element 36. A part or parts of the wick 37 are in fluid communication with aerosolizable material in the store 38 and by a wicking or capillary action aerosolizable material is drawn along or through the wick 37 to a part or parts of the wick 37 which are in contact with the heater 36.

Vaporization of the aerosolizable material occurs at the interface between the wick 37 and the heater 36 by the provision of heat energy to the aerosolizable material to cause evaporation, thus generating the aerosol. The aerosolizable material, the wick 37 and the heater 36 may be collectively referred to as an aerosol or vapour source.

The wick 37 and the heater 36 may be collectively referred to as a vaporizer or an atomiser 15.

As described above, the wick is formed a sintered material.

The sintered material may comprise sintered ceramic, sintered metal fibers/powders, or a combination of the two. Typically a single wick will be present, but it is envisaged that more than one wick could be present, for example, two, three, four or five wicks.

The (or at least one of/all of the) sintered wick(s) may have deposited thereon/embedded therein an electrically resistive heater. Such a heater may be formed from heat conducting alloys such as NiCr alloys. Alternatively, the sintered material may have such electrical properties such that when a current is passed there through, it is heated. Thus, the aerosol generating component and the wick may be considered to be integrated. In some embodiments, the aerosol generating component and the wick are formed from the same material and form a single component.

In some embodiments, the wick is formed from a sintered metal material and is generally in the form of a planar sheet. Thus, the wick element may have a substantially thin flat shape. For example it may be considered as a sheet, layer, film, substrate or the like. By this it is meant that a thickness of the wick is less or very much less than at least one of the length and the width of the wick. Thus, the wick thickness (its smallest dimension) is less or very much less than the longest dimension.

The wick may be made of a homogenous, granular, fibrous or flocculent sintered metal(s) so as to form said capillary structure. Wick elements can be made from a conductive material which is a nonwoven sintered porous web structure comprising metal fibres, such as fibres of stainless steel. For example, the stainless steel may be AISI (American Iron and Steel Institute) 316L (corresponding to European standard 1.4404). The material's weight may be in the range of 100-300 g/m².

Where the wick is generally planar, the thickness of the wick may be in the range of 75-250 μm. A typical fibre diameter may be about 12 μm, and a typical mean pore size (size of the voids between the fibres) may be about 32 μm. An example of a material of this type is Bekipor® ST porous metal fibre media manufactured by NV Bekaert SA, Belgium, being a range of porous nonwoven fibre matrix materials made by sintering stainless steel fibres.

Note also that while the material is described as planar, this refers to the relative dimensions of the sheet material and the wick (a thickness many times smaller than the length and/or width) but does not necessarily indicate flatness, in particular of the final wick made from the material. A wick may be flat but might alternatively be formed from sheet material into a non-flat shape such as curved, rippled, corrugated, ridged, formed into a tube or otherwise made concave and/or convex.

The wick element may have various properties. It is formed from a porous material to enable the required wicking or capillary effect for drawing source liquid through it from an store for aerosolizable material (where the wick meets the aerosolizable material at a store contact site) to the vaporisation interface. Porosity is typically provided by a plurality of interconnected or partially interconnected pores (holes or interstices) throughout the material, and open to the outer surface of the material. Any level of porosity may be employed depending on the material, the size of the pores and the required rate of wicking. For example a porosity of between 30% and 85% might be selected, such as between 40% and 70%, between 50% and 80%, between 35% and 75% or between 40% and 75%. This might be an average porosity value for the whole wick element, since porosity may or may not be uniform across the wick. For example, pore size at the store contact site might be different from pore size nearer to the heater.

It is useful for the wick to have sufficient rigidity to support itself in a required within the article. For example, it may be mounted at or near one or two edges and be required to maintain its position substantially without flexing, bending or sagging.

As an example, porous sintered ceramic is a useful material to use as the wick element. Any ceramic with appropriate porosity may be used. If porous ceramic is chosen as the porous wick material, this is available as a powder which can be formed into a solid by sintering (heating to cause coalescence, possibly under applied pressure). Sintering then solidifies the ceramic to create the porous wick.

The article 30 further includes a mouthpiece 35 having an opening through which a user may inhale the aerosol generated by the vaporizer 15. The aerosol for inhalation may be described as an aerosol stream or inhalable airstream.

The aerosol delivery device 20 includes a power source (a re-chargeable cell or battery 14, referred to herein after as a battery) to provide power for the e-cigarette 10, and a controller (printed circuit board (PCB)) 28 and/or other electronics for generally controlling the e-cigarette 10. The aerosol delivery device can therefore also be considered as a battery section, or a control unit or section.

In use, when the heater 36 receives power from the battery 14, as controlled by the circuit board 28 possibly in response to pressure changes detected by an air pressure sensor (not shown), the heater 36 vaporizes aerosolizable material delivered by the wick 37 to generate the aerosol, and this aerosol stream is then inhaled by a user through the opening in the mouthpiece 35. The aerosol is carried from the aerosol source to the mouthpiece 35 along an air channel (not shown in FIG. 1) that connects the aerosol source to the mouthpiece opening as a user inhales on the mouthpiece.

In this particular example, the device 20 and article 30 are detachable from one another by separation in a direction parallel to the longitudinal axis, as shown in FIG. 1, but are joined together when the system 10 is in use by cooperating engagement elements 21, 31 (for example, a screw, magnetic or bayonet fitting) to provide mechanical and electrical connectivity between the device 20 and the article 30, in particular connecting the heater 36 to the battery 14. The battery may be charged as is known to one skilled in the art.

In some embodiments, the article comprises/forms a sealed container. For example, the sealed container may be hermetically sealed. The present inventors have found that inclusion of the aerosolizable material in a sealed article assists in preventing water ingress into the system, which can prevent the cannabidiol from precipitating. The hermetically sealed container may comprise a blister pack with one or more hermetically sealed compartments for storage of one or more articles comprising the aerosolizable material described herein.

In some embodiments, the article comprises a housing within which the aerosolizable material is contained. The housing may be transparent such that the aerosolizable material can be viewed from outside of the housing. It may also be that the housing has a degree of opacity such that the passage of light through the housing is limited. This can be important so as to prevent light (such as ultra violet light) from entering the housing and compromising the stability of the aerosolizable material. In this regard, the present inventors have considered that cannabinoids may be particularly susceptible to such light destabilization. In some embodiments, the housing is formed from a material which inhibits/prevents the passage of ultra violet light there through. In some embodiments, it may be that the sealed container mentioned above is formed from a material which has a degree of opacity such that the passage of light through the sealed container is limited. Further, the sealed container mentioned above may be formed from a material which inhibits/prevents the passage of ultra violet light there through. This may be in addition to said sealed container being hermetically sealed and/or comprising a blister pack with one or more hermetically sealed compartments for storage of one or more articles comprising the aerosolizable material described herein.

In some embodiments, the aerosolizable material comprises at least one cannabinoid, and at least one carrier constituent, the material having a turbidity of about 10 NTU or less.

In this regard, the present inventors have found that when preparing an aerosolizable material comprising a cannabinoid, it is desirable to ensure that the turbidity of the material is 10 NTU or less. When the turbidity of the material is above this range, it is a sign that one or more of the constituents of the material is not present in the material in a stable manner. This could impact the use of the aerosolizable material in a number of ways. For example, the user may perceive the lack of stability and form an opinion that the aerosolizable material is of inferior quality. Alternatively or additionally, such instability may lead to inefficient transfer of one or more constituents from the aerosolizable material to the aerosol. Likewise, such instability may lead to the aerosolizable material causing suboptimal performance of any system or device using the material. The present inventors have found that issues of stability may be particularly pronounced when the aerosolizable material comprises a cannabinoid and have thus found that ensuring the aerosolizable material has a turbidity of 10 NTU or less is important.

In some embodiments, the turbidity of the aerosolizable material is about 10 NTU or less. In some embodiments, the turbidity of the aerosolizable material is about 9 NTU or less. In some embodiments, the turbidity of the aerosolizable material is about 8 NTU or less. In some embodiments, the turbidity of the aerosolizable material is about 7 NTU or less. In some embodiments, the turbidity of the aerosolizable material is about 6 NTU or less. In some embodiments, the turbidity of the aerosolizable material is about 5 NTU or less. In some embodiments, the turbidity of the aerosolizable material is about 4 NTU or less. In some embodiments, the turbidity of the aerosolizable material is about 3 NTU or less. In some embodiments, the turbidity of the aerosolizable material is about 2 NTU or less. In some embodiments, the turbidity of the aerosolizable material is about 1.5 NTU or less. In some embodiments, the turbidity of the aerosolizable material is about 1 NTU or less. In some embodiments, the turbidity of the aerosolizable material is about 0.9 NTU or less.

In some embodiments, the turbidity of the aerosolizable material is about 0.8 NTU or less.

In some embodiments, the turbidity of the aerosolizable material is about 0.7 NTU or less.

In some embodiments, the turbidity of the aerosolizable material is about 0.6 NTU or less.

In some embodiments, the turbidity of the aerosolizable material is about 0.5 NTU or less.

In some embodiments, the turbidity of the aerosolizable material is about 0.4 NTU or less.

In some embodiments, the turbidity of the aerosolizable material is about 0.3 NTU or less.

In some embodiments, the turbidity of the aerosolizable material is about 0.2 NTU or less.

In some embodiments, the turbidity of the aerosolizable material is from about 0.1 NTU to about 1 NTU. In some embodiments, the turbidity of the aerosolizable material is from about 0.2 NTU to about 1 NTU. In some embodiments, the turbidity of the aerosolizable material is from about 0.3 NTU to about 1 NTU. In some embodiments, the turbidity of the aerosolizable material is from about 0.4 NTU to about 1 NTU. In some embodiments, the turbidity of the aerosolizable material is from about 0.5 NTU to about 1 NTU. In some embodiments, the turbidity of the aerosolizable material is from about 0.1 NTU to about 0.9 NTU. In some embodiments, the turbidity of the aerosolizable material is from about 0.1 NTU to about 0.8 NTU. In some embodiments, the turbidity of the aerosolizable material is from about 0.1 NTU to about 0.7 NTU. In some embodiments, the turbidity of the aerosolizable material is from about 0.1 NTU to about 0.6 NTU. In some embodiments, the turbidity of the aerosolizable material is from about 0.1 NTU to about 0.5 NTU.

The turbidity of the aerosolizable material can be measured as is common in the art. For example, by using a TL2310 ISO Turbidimeter from Hach, Colorado, 80539-0389, United States.

Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In one embodiment, the cannabinoid is cannabidiol (CBD) or a pharmaceutically acceptable salt thereof. In one embodiment, the cannabidiol is synthetic cannabidiol. In one embodiment, the cannabidiol is added to the aerosolizable material in the form of an isolate. In one embodiment, the CBD is added to the aerosolizable material in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

The cannabinoid may be present in the aerosolizable material based on a mg/ml basis of the aerosolizable material.

In one embodiment, the cannabinoid is present in an amount of from about 5 mg/ml up to about 100 mg/ml. In one embodiment, the cannabinoid is present in an amount of from about 5 mg/ml up to about 90 mg/ml. In one embodiment, the cannabinoid is present in an amount of from about 5 mg/ml up to about 80 mg/ml. In one embodiment, the cannabinoid is present in an amount of from about 5 mg/ml up to about 70 mg/ml. In one embodiment, the cannabinoid is present in an amount of from about 5 mg/ml up to about 60 mg/ml. In one embodiment, the cannabinoid is present in an amount of from about 5 mg/ml up to about 50 mg/ml. In one embodiment, the cannabinoid is present in an amount of from about 5 mg/ml up to about 40 mg/ml. In one embodiment, the cannabinoid is present in an amount of from about 5 mg/ml up to about 30 mg/ml. In one embodiment, the cannabinoid is present in an amount of from about 5 mg/ml up to about 20 mg/ml. In one embodiment, the cannabinoid is present in an amount of from about 5 mg/ml up to about 10 mg/ml.

In one embodiment, the cannabinoid is present in an amount of about 5 mg/ml or more. In one embodiment, the cannabinoid is present in an amount of about 10 mg/ml or more. In one embodiment, the cannabinoid is present in an amount of about 15 mg/ml or more. In one embodiment, the cannabinoid is present in an amount of about 20 mg/ml or more. In one embodiment, the cannabinoid is present in an amount of about 25 mg/ml or more. In one embodiment, the cannabinoid is present in an amount of about 30 mg/ml or more. In one embodiment, the cannabinoid is present in an amount of about 35 mg/ml or more. In one embodiment, the cannabinoid is present in an amount of about 40 mg/ml or more. In one embodiment, the cannabinoid is present in an amount of about 45 mg/ml or more. In one embodiment, the cannabinoid is present in an amount of about 50 mg/ml or more. In one embodiment, the cannabinoid is present in an amount of about 55 mg/ml or more. In one embodiment, the cannabinoid is present in an amount of about 60 mg/ml or more. In one embodiment, the cannabinoid is present in an amount of about 65 mg/ml or more.

The carrier constituent comprises one or more constituents capable of forming an aerosol, particularly when evaporated and allowed to condense. In some embodiments, the carrier constituent may comprise one or more of glycerol, propylene glycol, triethylene glycol, tetraethylene glycol, 1,3-butylene glycol, erythritol, meso-Erythritol, ethyl vanillate, ethyl laurate, a diethyl suberate, triethyl citrate, triethylene glycol diacetate, triacetin, a diacetin mixture, benzyl benzoate, benzyl phenyl acetate, tributyrin, lauryl acetate, lauric acid, myristic acid, and propylene carbonate.

In one embodiment, the carrier constituent comprises propylene glycol.

In one embodiment, propylene glycol is present in an amount of from 10% w/w to 95% w/w based on the total weight of the material. In one embodiment, propylene glycol is present in an amount of from 20% w/w to 95% w/w based on the total weight of the material. In one embodiment, propylene glycol is present in an amount of from 30% w/w to 95% w/w based on the total weight of the material. In one embodiment, propylene glycol is present in an amount of from 40% w/w to 95% w/w based on the total weight of the material.

In one embodiment, propylene glycol is present in an amount of from 50% w/w to 90% w/w based on the total weight of the material. In one embodiment, propylene glycol is present in an amount of from 50% w/w to 85% w/w based on the total weight of the material. In one embodiment, propylene glycol is present in an amount of from 50% w/w to 80% w/w based on the total weight of the material. In one embodiment, propylene glycol is present in an amount of from 50% w/w to 75% w/w based on the total weight of the material. In one embodiment, propylene glycol is present in an amount of from 50% w/w to 60% w/w based on the total weight of the material. In one embodiment, propylene glycol is present in an amount of from 50% w/w to 65% w/w based on the total weight of the material. In one embodiment, propylene glycol is present in an amount of from 50% w/w to 60% w/w based on the total weight of the material.

In one embodiment, propylene glycol is present in an amount of from 55% w/w to 90% w/w based on the total weight of the material. In one embodiment, propylene glycol is present in an amount of from 60% w/w to 90% w/w based on the total weight of the material. In one embodiment, propylene glycol is present in an amount of from 65% w/w to 90% w/w based on the total weight of the material. In one embodiment, propylene glycol is present in an amount of from 70% w/w to 90% w/w based on the total weight of the material. In one embodiment, propylene glycol is present in an amount of from 75% w/w to 90% w/w based on the total weight of the material. In one embodiment, propylene glycol is present in an amount of from 80% w/w to 90% w/w based on the total weight of the material. In one embodiment, propylene glycol is present in an amount of from 85% w/w to 90% w/w based on the total weight of the material.

In one embodiment, propylene glycol is present in an amount of at least 10% w/w based on the total weight of the material. In one embodiment, propylene glycol is present in an amount of at least 20% w/w based on the total weight of the material. In one embodiment, propylene glycol is present in an amount of at least 30% w/w based on the total weight of the material. In one embodiment, propylene glycol is present in an amount of at least 40% w/w based on the total weight of the material. In one embodiment, propylene glycol is present in an amount of at least 50% w/w based on the total weight of the material. In one embodiment, propylene glycol is present in an amount of at least 55% w/w based on the total weight of the material. In one embodiment, propylene glycol is present in an amount of at least 60% w/w based on the total weight of the material. In one embodiment, propylene glycol is present in an amount of at least 65% w/w based on the total weight of the material. In one embodiment, propylene glycol is present in an amount of at least 70% w/w based on the total weight of the material. In one embodiment, propylene glycol is present in an amount of at least 75% w/w based on the total weight of the material. In one embodiment, propylene glycol is present in an amount of at least 80% w/w based on the total weight of the material. In one embodiment, propylene glycol is present in an amount of at least 85% w/w based on the total weight of the material. In one embodiment, propylene glycol is present in an amount of at least 90% w/w based on the total weight of the material.

In one embodiment, propylene glycol is present in an amount of about 70% w/w.

In some embodiments, the w/w % amount of propylene glycol in the material, based on the total weight of the material, is equal to or above a threshold C_{%}, the threshold being defined according to

C_{%}=11.416×(A)^{0.377}

wherein A is the amount of the at least one cannabinoid present in the material in mg/ml. It has been found that aerosolizable materials comprising at least one cannabinoid, such as cannabidiol, and propylene glycol conforming to the above threshold, are particularly stable.

In some embodiments, the amount of propylene glycol in the system is above the threshold C %. For example, the amount of propylene glycol may be about 1 w/w %, 2 w/w %, 3 w/w %, 4 w/w %, 5 w/w %, 6 w/w %, 7 w/w %, 8 w/w %, 9 w/w % or 10 w/w % above the threshold C_{%}. Including more propylene glycol relative to the threshold can be important if the aerosolizable material attracts water during storage. This additional propylene glycol can therefore prevent the CBD from precipitating during periods of storage.

In some embodiments, the aerosolizable material comprises less than 12% w/w water. In some embodiments, the aerosolizable material comprises less than 11% w/w water. In some embodiments, the aerosolizable material comprises less than 10% w/w water. In some embodiments, the aerosolizable material comprises less than 5% w/w water. In some embodiments, the aerosolizable material comprises less than 1% w/w water. In some embodiments, the aerosolizable material comprises less than 0.5% w/w water. In some embodiments, the aerosolizable material comprises substantially no water.

In one embodiment, the carrier constituent comprises glycerol.

In one embodiment, glycerol is present in an amount of from 10% w/w to 95% w/w based on the total weight of the material. In one embodiment, glycerol is present in an amount of from 20% w/w to 95% w/w based on the total weight of the material. In one embodiment, glycerol is present in an amount of from 30% w/w to 95% w/w based on the total weight of the material. In one embodiment, glycerol is present in an amount of from 40% w/w to 95% w/w based on the total weight of the material. In one embodiment, glycerol is present in an amount of from 50% w/w to 95% w/w based on the total weight of the material.

In one embodiment, glycerol is present in an amount of from 50% w/w to 90% w/w based on the total weight of the material. In one embodiment, glycerol is present in an amount of from 50% w/w to 85% w/w based on the total weight of the material. In one embodiment, glycerol is present in an amount of from 50% w/w to 80% w/w based on the total weight of the material. In one embodiment, glycerol is present in an amount of from 50% w/w to 75% w/w based on the total weight of the material. In one embodiment, glycerol is present in an amount of from 50% w/w to 60% w/w based on the total weight of the material. In one embodiment, glycerol is present in an amount of from 50% w/w to 65% w/w based on the total weight of the material. In one embodiment, glycerol is present in an amount of from 50% w/w to 60% w/w based on the total weight of the material.

In one embodiment, glycerol is present in an amount of from 55% w/w to 90% w/w based on the total weight of the material. In one embodiment, glycerol is present in an amount of from 60% w/w to 90% w/w based on the total weight of the material. In one embodiment, glycerol is present in an amount of from 65% w/w to 90% w/w based on the total weight of the material. In one embodiment, glycerol is present in an amount of from 70% w/w to 90% w/w based on the total weight of the material. In one embodiment, glycerol is present in an amount of from 75% w/w to 90% w/w based on the total weight of the material. In one embodiment, glycerol is present in an amount of from 80% w/w to 90% w/w based on the total weight of the material. In one embodiment, glycerol is present in an amount of from 85% w/w to 90% w/w based on the total weight of the material.

In one embodiment, glycerol is present in an amount of at least 10% w/w based on the total weight of the material. In one embodiment, glycerol is present in an amount of at least 20% w/w based on the total weight of the material. In one embodiment, glycerol is present in an amount of at least 30% w/w based on the total weight of the material. In one embodiment, glycerol is present in an amount of at least 40% w/w based on the total weight of the material. In one embodiment, glycerol is present in an amount of at least 50% w/w based on the total weight of the material. In one embodiment, glycerol is present in an amount of at least 50% w/w based on the total weight of the material. In one embodiment, glycerol is present in an amount of at least 55% w/w based on the total weight of the material. In one embodiment, glycerol is present in an amount of at least 60% w/w based on the total weight of the material. In one embodiment, glycerol is present in an amount of at least 65% w/w based on the total weight of the material. In one embodiment, glycerol is present in an amount of at least 70% w/w based on the total weight of the material. In one embodiment, glycerol is present in an amount of at least 75% w/w based on the total weight of the material. In one embodiment, glycerol is present in an amount of at least 80% w/w based on the total weight of the material. In one embodiment, glycerol is present in an amount of at least 85% w/w based on the total weight of the material. In one embodiment, glycerol is present in an amount of at least 90% w/w based on the total weight of the material.

In one embodiment, both glycerol and propylene glycol are present as carrier constituents.

In one embodiment, glycerol and propylene glycol are present in the aerosolizable material in the following amounts:
60 to 90% w/w propylene glycol; and
40 to 10% w/w glycerol, based on the total weight of glycerol and propylene glycol present in the material.

In one embodiment, glycerol and propylene glycol are present in the aerosolizable material in the following amounts:
70 to 80% w/w propylene glycol; and
30 to 20% w/w glycerol.
based on the total weight of glycerol and propylene glycol present in the material.

In one embodiment, the aerosolizable material comprises about 70% w/w propylene glycol and about 30% glycerol.

In one embodiment, the aerosolizable material is a liquid at about 25° C.

The aerosolizable material may comprise one or more further constituents. In particular, one or more further constituents may be selected from one or more physiologically and/or olfactory active constituents, and/or one or more functional constituents.

In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

In some embodiments, the active constituent is an olfactory active constituent and may be selected from a “flavor” and/or “flavorant” which, where local regulations permit, may be used to create a desired taste, aroma or sensation in a product for adult consumers. In some instances such constituents may be referred to as flavors, flavorants, cooling agents, heating agents, or sweetening agents, and may include one or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

The flavor may be added to the aerosolizable material as part of a so-called “flavor block”, where one or more flavors are blended together and then added to the aerosolizable material.

In some embodiments, the olfactory active constituent comprises a terpene. In some embodiments, the terpene is a terpene derivable from a phytocannabinoid producing plant, such as a plant from the stain of the Cannabis sativa species, such as hemp. In some embodiments, the aerosolizable material comprises a cannabinoid isolate in combination with a terpene derivable from a phytocannabinoid producing plant.

Suitable terpenes in this regard include so-called “C10” terpenes, which are those terpenes comprising 10 carbon atoms, and so-called “C15” terpenes, which are those terpenes comprising carbon atoms.

In some embodiments, the aerosolizable material comprises more than one terpene. For example, the aerosolizable material may comprise one, two, three, four, five, six, seven, eight, nine, ten or more terpenes as defined herein.

In some embodiments, the terpene is selected based on its solubility in a propylene glycol/glycerol system.

In this regard, the terpene may be selected on the basis of being soluble when present in a propylene glycol/glycerol system, where the w/w % amount of propylene glycol C % present in the material, based on the total weight of the material, is determined on the basis of the following relationship:

C_{%}=11.416×(T)^{0.377}

wherein T is the amount of the at least one terpene present in the material in mg/ml.

By ensuring the selected terpene meets the above threshold when present in a propylene glycol/glycerol system, the stability of the system will not be substantially compromised by including a terpene. In other words, the terpene(s) may be selected such that their solubility in propylene glycol is substantially matched to that of cannabidiol.

In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

In some embodiments, the aerosolizable material comprises a combination of terpenes. In some embodiments, the combination of terpenes may comprise a combination of at least geraniol and linalool. In some embodiments, the combination of terpenes may comprise a combination of at least eucalyptol and menthone. In some embodiments, the combination of terpenes may comprise a combination of at least eucalyptol, carvone, piperitone and menthone. In some embodiments, the combination of terpenes may comprise a combination of at least eucalyptol, carvone, beta-bourbonene, germacrene, piperitone, iso-menthone and menthone.

In one embodiment, the terpene(s) are present in a flavour block. This means that the terpenes are blended with one or more other flavours (optionally with an appropriate solvent, for example propylene glycol) and then the flavour block is added during the manufacture of the aerosolizable material. In some embodiments, the total amount of the flavour block present in the aerosolizable material is up to about 10 w/w %. In some embodiments, the total amount of the flavour block present in the aerosolizable material is up to about 9 w/w %. In some embodiments, the total amount of the flavour block present in the aerosolizable material is up to about 8 w/w %. In some embodiments, the total amount of the flavour block present in the aerosolizable material is up to about 7 w/w %. In some embodiments, the total amount of the flavour block present in the aerosolizable material is up to about 6 w/w %. In some embodiments, the total amount of the flavour block present in the aerosolizable material is up to about 5 w/w %.

In one embodiment, the total amount of terpene present in the aerosolizable material is up to about 10 mg/ml. In one embodiment, the total amount of terpene present in the aerosolizable material is up to about 9 mg/ml. In one embodiment, the total amount of terpene present in the aerosolizable material is up to about 8 mg/ml. In one embodiment, the total amount of terpene present in the aerosolizable material is up to about 7 mg/ml. In one embodiment, the total amount of terpene present in the aerosolizable material is up to about 6 mg/ml. In one embodiment, the total amount of terpene present in the aerosolizable material is up to about 5 mg/ml. In one embodiment, the total amount of terpene present in the aerosolizable material is up to about 4 mg/ml. In one embodiment, the total amount of terpene present in the aerosolizable material is up to about 3 mg/ml. In one embodiment, the total amount of terpene present in the aerosolizable material is up to about 2 mg/ml. In one embodiment, the total amount of terpene present in the aerosolizable material is up to about 1 mg/ml.

In one embodiment, the total amount of terpene present in the aerosolizable material is from about 0.1 mg/ml up to about 10 mg/ml. In one embodiment, the total amount of terpene present in the aerosolizable material is from about 0.2 mg/ml up to about 10 mg/ml. In one embodiment, the total amount of terpene present in the aerosolizable material is from about 0.3 mg/ml up to about 10 mg/ml. In one embodiment, the total amount of terpene present in the aerosolizable material is from about 0.4 mg/ml up to about 10 mg/ml. In one embodiment, the total amount of terpene present in the aerosolizable material is from about 0.5 mg/ml up to about 10 mg/ml. In one embodiment, the total amount of terpene present in the aerosolizable material is from about 1.0 mg/ml up to about 10 mg/ml. In one embodiment, the total amount of terpene present in the aerosolizable material is from about 2.0 mg/ml up to about 10 mg/ml. In one embodiment, the total amount of terpene present in the aerosolizable material is from about 3.0 mg/ml up to about 10 mg/ml. In one embodiment, the total amount of terpene present in the aerosolizable material is from about 4.0 mg/ml up to about 10 mg/ml. In one embodiment, the total amount of terpene present in the aerosolizable material is from about 5.0 mg/ml up to about 10 mg/ml.

In one embodiment, the total amount of terpene present in the aerosolizable material is from about 0.1 mg/ml up to about 9.0 mg/ml. In one embodiment, the total amount of terpene present in the aerosolizable material is from about 0.1 mg/ml up to about 8.0 mg/ml. In one embodiment, the total amount of terpene present in the aerosolizable material is from about 0.1 mg/ml up to about 7.0 mg/ml. In one embodiment, the total amount of terpene present in the aerosolizable material is from about 0.1 mg/ml up to about 6.0 mg/ml. In one embodiment, the total amount of terpene present in the aerosolizable material is from about 0.1 mg/ml up to about 5.0 mg/ml. In one embodiment, the total amount of terpene present in the aerosolizable material is from about 0.1 mg/ml up to about 1 mg/ml. In one embodiment, the total amount of terpene present in the aerosolizable material is from about 0.1 mg/ml up to about 0.9 mg/ml. In one embodiment, the total amount of terpene present in the aerosolizable material is from about 0.1 mg/ml up to about 0.8 mg/ml. In one embodiment, the total amount of terpene present in the aerosolizable material is from about 0.1 mg/ml up to about 0.7 mg/ml. In one embodiment, the total amount of terpene present in the aerosolizable material is from about 0.1 mg/ml up to about 0.6 mg/ml. In one embodiment, the total amount of terpene present in the aerosolizable material is from about 0.1 mg/ml up to about 0.5 mg/ml.

For the avoidance of doubt, combinations of the above end points are explicitly envisaged by the present disclosure. This applies to any of the ranges disclosed herein.

The one or more other functional constituents may comprise one or more of pH regulators, coloring agents, preservatives, binders, fillers, stabilizers, and/or antioxidants. In particular, the pH regulator may include one or more acids selected from organic or inorganic acids. An example of an inorganic acid is phosphoric acid. The organic acid may include a carboxylic acid. The carboxylic acid may be any suitable carboxylic acid. In one embodiment the acid is a mono-carboxylic acid. In one embodiment the acid may be selected from the group consisting of acetic acid, lactic acid, formic acid, citric acid, benzoic acid, pyruvic acid, levulinic acid, succinic acid, tartaric acid, oleic acid, sorbic acid, propionic acid, phenylacetic acid, and mixtures thereof.

In some embodiments, an acceptable turbidity is achieved without the use of functional constituents which influence the stability of the aerosolizable material. For example, it may be possible to decrease the turbidity of a liquid system by introducing surface active constituents which serve to improve the emulsification/dispersion of one or more of the constituents. However, it may not be desirable to include such functional constituents due to user acceptability. Therefore, in some embodiments, the aerosolizable material does not comprise a surface active constituent. Examples of surface active constituents include medium chain triglycerides (MCT) and tocopherol acetate.

In some embodiments, an acceptable turbidity is achieved without the use of any/significant amounts of water. In this regard, whilst water may otherwise assist in the preparation of aerosolizable materials since water containing materials may have a lower viscosity and therefore may be transferred more easily to an aerosol generating component, it has been found in the context of the present disclosure that water can negatively influence the stability of the aerosolizable material containing at least one cannabinoid.

In some embodiments, the aerosolizable material comprises less than 12% w/w water. In some embodiments, the aerosolizable material comprises less than 11% w/w water. In some embodiments, the aerosolizable material comprises less than 10% w/w water. In some embodiments, the aerosolizable material comprises less than 5% w/w water.

In some embodiments, the aerosolizable material comprises less than 1% w/w water.

In some embodiments, the aerosolizable material comprises less than 0.5% w/w water. In some embodiments, the aerosolizable material comprises substantially no water.

In particular, it has been found that for certain aerosolizable materials comprising a cannabinoid, such as cannabidiol, if the material comprises water in amounts of about 12% w/w, the cannabinoid is rendered unstable.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An article for use with an aerosol provision system, the article comprising a store comprising an aerosolizable material and a wick formed from a sintered material, wherein the aerosolizable material comprises at least one cannabinoid and at least one carrier constituent.

2. The article according to claim 1, wherein the sintered material is a ceramic material.

3. The article according to claim 1, wherein the sintered material comprises metal fibers.

4. The article according to claim 1, wherein article comprises an aerosol generating component.

5. The article according to claim 4, wherein the aerosol generating component and the wick are integrated as a single component.

6. The article according to claim 4, wherein the aerosol generating component and the wick are separate components.

7. The article according to claim 1, wherein the formulation has a turbidity of about 10 NTU or less.

8. The article according to claim 1, wherein the carrier constituent comprises one or more of the group consisting of: glycerine, glycerol, propylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

9. The article according to claim 8, wherein the carrier constituent comprises propylene glycol.

10. The article according to claim 9, wherein propylene glycol is present in an amount of at least 50% w/w based on the total weight of the material.

11. The article according to claim 10, wherein propylene glycol is present in an amount of at least 60% w/w based on the total weight of the material.

12. The article according to claim 11, wherein propylene glycol is present in an amount of at least 70% w/w based on the total weight of the material.

13. The article according to claim 8, wherein herein the carrier constituent comprises glycerol.

14. The article according to claim 13, wherein glycerol is present in an amount of at least 50% w/w based on the total weight of the material.

15. The article according to claim 14, wherein glycerol is present in an amount of at least 60% w/w based on the total weight of the material.

16. The article according to claim 15, wherein glycerol is present in an amount of at least 70% w/w based on the total weight of the material.

17. The article according to claim 8, wherein both glycerol and propylene glycol are present as carrier constituents.

18. The article according to claim 17, wherein, based on the total amount of propylene glycol and glycerol in the aerosolizable material, the aerosolizable material comprises:
60 to 90% w/w propylene glycol; and
to 10% w/w glycerol.

19. The article according to claim 18, wherein, based on the total amount of propylene glycol and glycerol in the aerosolizable material, the aerosolizable material comprises:
70 to 80% w/w propylene glycol; and
to 20% w/w glycerol.

20. The article according to claim 19, wherein, based on the total amount of propylene glycol and glycerol in the aerosolizable material, the aerosolizable material comprises about 70% w/w propylene glycol and about 30% glycerol.

21. The article according to claim 1, wherein the cannabinoid is cannabidiol.

22. The article according to claim 1, wherein the cannabinoid is present in an amount of 5 mg/ml of material or more.

23. The article according to claim 22, wherein the cannabinoid is present in the material in an amount of 10 mg/ml of material or more.

24. The article according to claim 22, wherein the cannabinoid is present in the material in an amount of 30 mg/ml of material or more.

25. The article according to claim 22, wherein the cannabinoid is present in the material in an amount of 60 mg/ml of material or more.

26. The article according to claim 1, wherein the material takes the form of a liquid at 25° C.

27. The article according to claim 1, wherein the material further comprises one or more active constituents in addition to the cannabinoid.

28. The article according to claim 27, wherein the one or more active constituents is an olfactory active constituent.

29. An aerosol provision system comprising an aerosol provision device and an article as defined in claim 1.
